(19) United States
(12) Patent Application Publication
(10) Pub. No.: US 2013/0172694 A1
(43) Pub. Date: Jul. 4, 2013

(54) SWALLOWABLE CAPSULE FOR MONITORING A CONDITION

(71) Applicants: Hans Zou, Windsor, NJ (US); Jeff Shimizu, Cortlandt Manor, NY (US)

(72) Inventors: Hans Zou, Windsor, NJ (US); Jeff Shimizu, Cortlandt Manor, NY (US)

(73) Assignee: MEDIMETRICS PERSONALIZED DRUG DELIVERY B.V., Eindhoven (NL)

(21) Appl. No.: 13/516,356

(22) PCT Filed: Dec. 14, 2010
(86) PCT No.: PCT/IB2010/055790

§ 371 Date: Jan. 25, 2013

Related U.S. Application Data
(60) Provisional application No. 61/287,363, filed on Dec. 17, 2009.

(51) Int. Cl. A61B 5/07; A61B 5/145; A61B 5/155; A61B 5/01; A61B 5/053; A61B 5/157; A61B 5/00; A61B 5/03

(52) U.S. Cl. CPC A61B 5/07; A61B 5/4839; A61B 5/14539; A61B 5/036; A61B 5/14546; A61B 5/01; A61B 5/0538; A61B 5/4848; A61B 5/157; A61B 5/155; A61B 5/7246; A61B 5/0008
USPC 600/302

(57) ABSTRACT

A swallowable capsule (10) is provided with a drug reservoir (18) and at least one sensor (13). The drug reservoir (18) is arranged to hold a pharmaceutical composite. A wall (12) of the drug reservoir (18) comprises at least one opening for allowing fluid from the surroundings of the capsule (10) to enter the drug reservoir (18) and for allowing dissolved drug or liberated particles of the pharmaceutical composite to enter the surroundings of the capsule (10). The at least one sensor (13) is provided for monitoring a condition of the surroundings of the capsule (10).

### FIELD OF THE INVENTION

This invention relates to a swallowable capsule comprising at least one sensor for monitoring a condition of the surroundings of the capsule.

### BACKGROUND OF THE INVENTION

In recent years swallowable capsules with electronic components have been developed for mainly two purposes. Miniature swallowable sensors are used for monitoring environmental conditions inside the gastrointestinal tract of animal and human beings. In addition, active drug delivery devices are available for enabling controlled delivery of a drug dosage at specific positions inside the gastrointestinal tract. In some swallowable drug delivery devices, sensors are included for controlling the drug delivery based on sensed conditions.

To develop a proper drug formation or dosage form, it is important to obtain good correlation between in-vitro and in-vivo test data. While in-vitro tests conditions are always under control, in-vivo conditions can only be guessed based on statistics. Lack of real-time in-vivo data about gastrointestinal conditions of test-subjects forces investigators to work with a very large sample base to compensate for random deviation. It is expensive, time-consuming and usually not repeatable.

With advance in telemetry medicine, it is possible to monitor gastrointestinal conditions of test subjects in real-time. In clinical trials, sensor capsules are sometimes swallowed at the same moment as a drug to be tested. The sensor capsule is then used for monitoring, e.g., the pH of the gastrointestinal tract of patients having swallowed the drug. In this way, the sensor capsules may provide useful information about the functioning of the tested drug. One of the problems of the use of sensor capsules for monitoring the effect of a drug is that the drug and the sensor capsule may not traverse the gastrointestinal tract at the same pace. Therefore, the sensed parameters may not provided information about the direct surroundings of the drug.

### OBJECT OF THE INVENTION

It is an object of the invention to provide a sensor capsule which is capable of following a drug more closely.

### SUMMARY OF THE INVENTION

According to a first aspect of the invention, this object is achieved by providing a swallowable capsule, comprising a drug reservoir for holding a pharmaceutical composite with a dissolvable drug, a wall of the drug reservoir comprising at least one opening for allowing fluid from the surroundings of the capsule to enter the drug reservoir and for allowing liberated particles of the dissolvable drug to enter the surroundings of the capsule, and at least one sensor for monitoring a condition of the surroundings of the capsule.

By combining the drug delivery and the sensor functionality in one capsule, it is ensured that the sensor and the drug traverse the gastrointestinal tract together. The sensor will continuously be able to monitor the direct environment of the drug. This makes the capsule very suitable for studying drug release, dissolution and absorption properties. The opening in the wall of the drug reservoir allows environmental fluid to enter the drug reservoir and to transport the drug into the capsule surroundings.

The dissolving of the drug may take place inside the drug reservoir, resulting in dissolved drugs passing through the opening or openings in the reservoir wall. A gradual dissolving of the pharmaceutical composite then causes the drug to be released somewhere along the path of the capsule through the intestinal tract. Alternatively, drug particles which are gradually liberated or disintegrated from the pharmaceutical composite may be small enough to be able to pass the opening or openings in the reservoir wall without being dissolved immediately. The liberation of the drug particles may be initiated or caused by contact with the environmental fluid and is strongly affected by the conditions of the environmental fluid. The liberated drug particles may then dissolve somewhere in the intestinal tract, after having passed the reservoir wall of the capsule.

Because of the permeable wall of the drug reservoir, the drug uptake is realized in the same manner as if it would have been swallowed separate from the sensor capsule. Fluid flowing into and out of the drug reservoir interacts with the pharmaceutical composite in a way similar to when it would have been swallowed separately. A big advantage of the swallowable capsule according to the invention is that it monitors environmental conditions of the administered drug without interfering with its in-vivo interaction and does not need the drug to be in a special form suitable for such a drug delivery mechanism. The drug can be placed inside the reservoir in the same form as in which it is used when administering the drug without any accompanying sensors.

In a preferred embodiment, the drug reservoir can be (temporarily) opened or detached from the capsule in order to allow placement of the pharmaceutical composite in the reservoir. In such an embodiment, the same capsule can be used for monitoring the effects of different drugs. The drug does not have to be inserted into the drug reservoir during the production of the capsule. The drug reservoir may be loaded with the appropriate drug in a particular dosage form just before use.

In a special embodiment of the capsule according to the invention, the drug reservoir comprises a meshed wall, the meshed wall comprising the at least one opening. The dimensions of the meshed wall may be adapted to the drug dosage form with which the capsule is intended to be used. Different meshes may be provided for use with different drug dosage forms.

These and other aspects of the invention are apparent from and will be elucidated with reference to the embodiments described hereinafter.

### BRIEF DESCRIPTION OF THE DRAWINGS

In the drawings:

FIG. 1 schematically shows a swallowable capsule according to the invention, and

FIG. 2 schematically shows an opened capsule according to the invention.

### DETAILED DESCRIPTION OF THE INVENTION

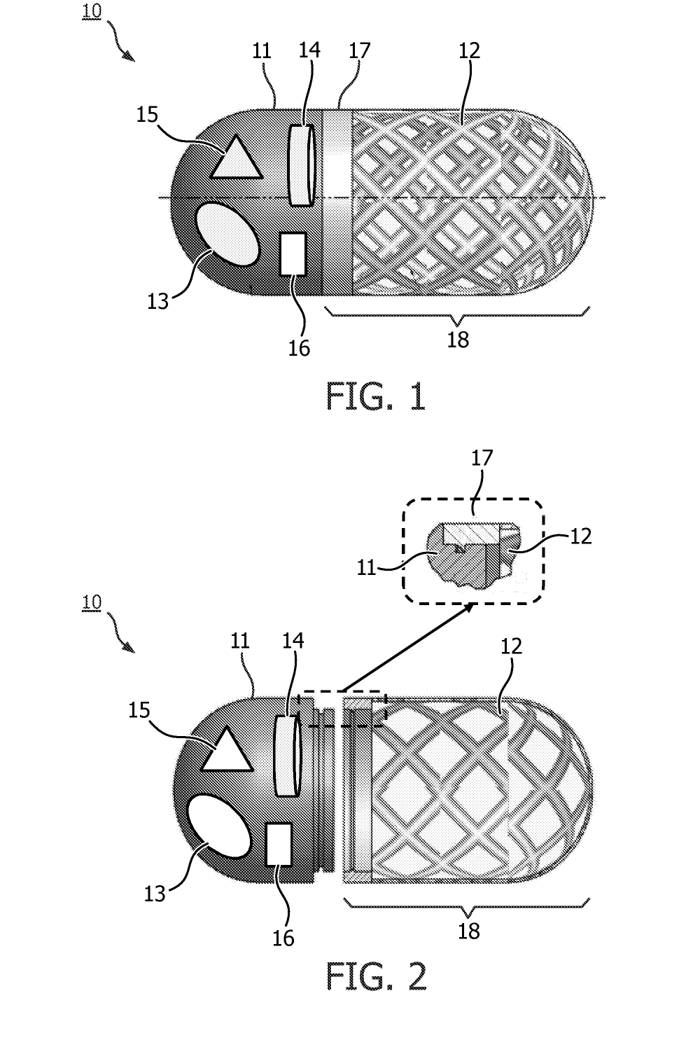

FIG. 1 schematically shows a swallowable capsule 11 according to the invention. The capsule 10 comprises two modules, a drug module 18 and an electronics module 11 which are preferably separable and coupled by a coupling means 17. The preferred diameter of the capsule 10 is less than 20 mm, even more preferred less than 15 mm, and the preferred total length is less than 30 mm. Larger capsules 10 may be difficult to swallow and/or may have problems with traversing the gastrointestinal tract.

The drug module 18 comprises a drug reservoir with a meshed side wall 12 which allows fluid from the environment to flow into and out of the drug reservoir. In this embodiment a meshed side wall 12 is shown, but in principle any other wall with one or more large enough openings to allow fluid flowing through may be used. The meshed side wall 12 may be injection molded or mad of thin metal wires, optionally a combination of both. Mesh opening size can be different depending on the shape, type or size of the drug dosage forms. The size of the opening or openings in the reservoir wall is preferably dimensioned in such a way that interaction between the drug and the environment is not hindered. Such a side wall is substantially open to the surroundings while physically holding the drug in place.

The drug may, e.g., be in a tablet, capsule, granules or other usual dosage form which optionally incorporates an enteric coating, polymer matrix or other means to control drug release rate. In addition to one or more active ingredients, the drug may comprise one or more excipients in order to make the drug available in a swallowable and stable dosage form. The drug reservoir may be filled with more than one tablet and/or capsule for simultaneous delivery of different drugs or to increase the dose. Interaction with the environmental fluid coming through the opening or openings in the reservoir wall may cause the drug to be dissolved. Alternatively, small drug particles are gradually liberated from the drug tablet or capsule and the environmental fluid causes the liberated drug particles to be transported out of the drug reservoir into the environment of capsule 10, where the drug will dissolve later.

An electronics module 11 comprises at least one sensor 13, coupled to an energy source 14, e.g. a battery, for powering the sensor 13. The sensor may, e.g., be a pH sensor, a pressure sensor, an ion sensor, an enzyme sensor, a temperature sensor, a blood sensor or an impedance sensor. Sensor data may be stored in a memory 16 and/or transmitted to an external receiver (not shown) by a data transmitter 15. The sensor 10 is optionally capable of producing time-stamped information relating to properties of the gastrointestinal fluid nearby the capsule 10. The properties of the environment of the capsule 10 are determined by the physiological condition of the subject (he who has swallowed the capsule) and the effects of the drug thereon. The possibility to monitor the disintegration, dissolving and absorption (coordinated with pharmacokinetic study) of a drug in-vivo and in real time, makes this drug carrier device 10 very suitable for application in drug development and clinical trials.

FIG. 2 schematically shows an opened capsule 10 according to the invention. In this embodiment, the capsule 10 consists of two separate modules 11, 18. Alternatively, the capsule 10 may be a singly body, with an opening for introducing the drug into the drug reservoir. It is also possible to already add the drug during the production of the capsule 10, but that may limit the possibilities of using the capsule 10 for different types of drugs or different drug doses.

Each module 11, 18 comprises part of the coupling means 17 for coupling the drug module 18 to the electronics module 11. The coupling means 17 are preferably such that the capsule 10 can be opened and closes repeatedly. It is however also possible to provide the capsule 10 as two separate modules 11, 18 and only allow the coupling means to join those modules 11, 18 together once, after filling the drug module 18 with the appropriate drug. The coupling means 17 may, e.g., use a threaded coupling part for screwing the modules 11, 18 together. The capsule 10 shown in FIG. 2 uses a complementary hooking structure to facilitate connection of the two modules 11, 18. The modules 11, 18 of the capsule 10 shown are easily clicked together. A close up of this complementary hooking structure 17 in a connected state is also shown in the Figure.

The invention also relates to a method to conduct a pharmacokinetic study on a drug in a pharmaceutical composite using a test subject, wherein a swallowable capsule 10 according to the invention is used to study the blood plasma drug concentration as a function of a condition of the surroundings of the capsule 10, the method comprising the following steps:
a. loading the drug reservoir 18 of the swallowable capsule 10 with the pharmaceutical composite;
b. administering the swallowable capsule 10 to the test subject;
c. sensing the condition of the surroundings of the capsule continuously by the sensor 13, resulting in time-stamped condition data;
d. taking a series of blood samples from the subject at different temporal points with reference to the time when the swallowable capsule 10 is administered for analysis of the blood concentration of the drug;
e. measuring the blood concentration resulting in time-stamped blood concentration data, and
f. comparing the time-stamped blood concentration data with the time-stamped condition data.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A swallowable capsule (10), comprising:
a drug reservoir (18) for holding a pharmaceutical composite, with a wall (12) comprising at least one opening for allowing fluid from the surroundings of the capsule (10) to enter the drug reservoir (18) and for allowing dissolved drug or liberated particles of the pharmaceutical composite to enter the surroundings of the capsule (10), and
at least one sensor (13) for monitoring a condition of the surroundings of the capsule (10).

2. A swallowable capsule (10) as claimed in claim 1, wherein the drug reservoir (18) can be opened or detached from the capsule (10) in order to allow placement of the pharmaceutical composite in the reservoir (18).

3. A swallowable capsule (10) as claimed in claim 1, wherein the drug reservoir (18) comprises a meshed wall (12), the meshed wall (12) comprising the at least one opening.

4. A swallowable capsule (10) as claimed in claim 3, wherein the meshed wall (12) is injection molded.

5. A swallowable capsule (10) as claimed in claim 3, wherein the meshed wall (12) comprises meshed metal wires.

6. As swallowable capsule (10) as claimed in claim 1, wherein the sensor (13) is capable of providing time-stamped data.

7. A swallowable capsule (10) as claimed in claim 1, further comprising a transmitter (15) coupled to the sensor (13) for transmitting sensor information regarding the monitored condition to an external receiver.

8. A swallowable capsule (10) as claimed in claim 1, wherein the at least one sensor (13) is a pH sensor, a pressure sensor, an ion sensor, an enzyme sensor, a temperature sensor, a blood sensor or an impedance sensor.

9. A method to conduct a pharmacokinetic study on a drug in a pharmaceutical composite using a test subject, wherein a swallowable capsule (10) according to claim 6 is used to study the blood plasma drug concentration as a function of a condition of the surroundings of the capsule (10), the method comprising the following steps:
a. loading the drug reservoir (18) of the swallowable capsule (10) with the pharmaceutical composite;
b. administering the swallowable capsule (10) to the test subject;
c. sensing the condition of the surroundings of the capsule continuously by the sensor (13), resulting in time-stamped condition data;
d. taking a series of blood samples from the subject at different temporal points with reference to the time when the capsule is administered for analysis of the blood concentration of the drug,
e. measuring the blood concentration resulting in time-stamped blood concentration data; and
f. comparing the time-stamped blood concentration data with the time-stamped condition data.
